(19) United States
(12) Patent Application Publication
(10) Pub. No.: US 2016/0030622 A1
(43) Pub. Date: Feb. 4, 2016

(54) Multiple Plasma Driven Catalyst (PDC) Reactors

(71) Applicant: Nano and Advanced Materials Institute Limited, Hong Kong (HK)

(72) Inventors: Jifan LI, Hong Kong (HK); Lei WANG, Hong Kong (HK); Ka Chun LEE, Hong Kong (HK); Wai Yan CHAN, Hong Kong (HK)

(21) Appl. No.: 14/592,916

(22) Filed: Jan. 9, 2015

Related U.S. Application Data
(63) Continuation-in-part of application No. 14/446,311, filed on Jul. 29, 2014, now Pat. No. 9,138,504.
(60) Provisional application No. 62/078,940, filed on Nov. 12, 2014.

(51) Int. Cl. A61L 9/22

(52) U.S. Cl. CPC A61L 9/22

(57) ABSTRACT

The present invention relates to an air purification system with plasma driven catalyst technology. The system comprises a plurality of plasma driven catalyst reactors in a parallel setup. Zigzag electrodes comprising zigzag structures are used to facilitate plasma generation. The system of the present invention provides the advantages with ease of fabrication and scaling-up, and improvement of air purification performance.

### CROSS REFERENCE TO RELATED APPLICATION

This is a continuation-in-part application of the non-provisional patent application Ser. No. 14/446,311 filed Jul. 29, 2014, and the disclosure of which is incorporated herein by reference in its entirety. Pursuant to 35 U.S.C.§119(e), this is a non-provisional patent application which claims benefit from U.S. provisional patent application Ser. No. 62/078,940 filed Nov. 12, 2014, and the disclosure of which is incorporated herein by reference.

### FIELD OF THE INVENTION

The present invention relates to the use of plasma driven catalyst (PDC) technology for disinfecting, cleaning and purifying air. More particularly, the present invention relates to an air purification system with multiple plasma driven catalyst reactors in a parallel setup.

### BACKGROUND

According to various researches, there is about 80% of the time in a day that urban population spends in an indoor environment, such as homes, offices, cinemas, restaurants, stores, and other semi-enclosed spaces. The air quality in these semi-enclosed spaces may be poor due to a variety of indoor air pollutant sources such as smoking, cooking fumes, wallpaper glue, coils, burning oil heaters, smog, PM2.5, etc. Continuous or intermittent discharge of various pollutants can cause severe human health damage and even death. Maintaining appropriate indoor air quality by ventilation often draws outdoor air to dilute indoor air space. However, if the outdoor air quality is poor or there is a lack of capacity for indoor ventilation, indoor air pollution continues. Thus, an air purification system is needed to purify the indoor air so as to improve the air quality.

Plasma is known to be a gas with ionized molecules, which contains a number of components like electrons of different energy, positive and negative ions, and neutral particles. Many studies have shown that plasma is attractive for removal of NOx, SOx, odors and VOCs. Among various types of plasma, non-thermal plasma has been demonstrated as a quite effective technology to decompose VOCs and other air pollutants. The non-thermal plasma has some unique properties such as quick response at ambient temperature and under atmospheric pressure, achievement of high electron energies within short residence times, and easy operations. Besides, the plasma discharge works like an electrostatic precipitator and can be used for dust and liquid droplet collection. There is, however, a consensus among researchers that application of plasma for VOC abatement suffers from 3 main weaknesses, i.e. incomplete oxidation with emission of harmful compounds (CO, NOx, other VOCs), a poor energy efficiency, and a low mineralization degree. The incomplete oxidation leads to the formation of toxic by-products such as carbon monoxide (CO), ozone and aerosol particles, which even increase the total gaseous toxicity. Thus, these by-products formation requires the additional post-treatment system, which increases the cost and complexity of the whole air purifying system.

EP1671659 discloses a disinfecting and purifying apparatus comprising: a casing; an orientation air deflector, disposed on said casing; a movable air deflector, included in said casing and disposed at a position corresponding to said orientation air deflector; a plasma reactor, installed below said movable air deflector, and said plasma reactor installs an anion anode plate, an anion cathode plate, a plasma anode plate and a plasma cathode plate sequentially from top to bottom, and said anion anode plate, anion cathode plate, plasma anode plate and plasma cathode plate are meshed stainless steel plates, and a thin film of nano catalyst is coated on the surface of said meshed stainless steel plate. Nevertheless, using such method to generate plasma is not effective in providing a plasma with high intensity due to the catalyst coating on the anode and cathode plates.

According to a conventional air purification apparatus with the combination of plasma and catalyst for air treatment. Plasma is generated within a chamber, and a photocatalyst layer is place at the air outlet of the chamber, or at the air inlet of the chamber. Nevertheless, such configuration is not effective in air purification since the photocatalyst layer generates high air resistance toward the air purification system, leading to the attenuation of air circulation rate and the increase of the electrical burden towards fans. What's more, an additional UV light lamp is required to irradiate the photocatalyst layer for free radical generation that increases the cost and the complexity of the air purification system. Also simply applying UV irradiation may not be strong enough to generate sufficient free radicals to decompose air pollutants, ultimately causing ineffective air purification.

Consequently, there is an unmet need to have an air purification apparatus, which is effective in air pollutant removal with low air resistance, system complexity, and power consumption.

### SUMMARY OF THE INVENTION

The present invention relates to an air purification system with plasma driven catalyst technology. The system comprises a plurality of plasma driven catalyst reactors in a parallel setup.

According to an embodiment of the presently claimed invention, an air purification system, comprises: a plurality of plasma driven catalyst (PDC) reactors in a parallel setup; wherein the PDC reactors are stacked in parallel with each other; wherein each of the PDC reactors comprises a first electrode, a second electrode, a first dielectric layer, and a first photocatalytic layer; wherein the first electrode and the second electrode are located in parallel with each other, and the first dielectric layer is placed between the first electrode and the second electrode and formed on the first electrode; wherein the first photocatalytic coating is coated on the second electrode or the first dielectric layer.

Preferably, the each of the PDC reactors is rectangular or square in shape for facilitating stacking among the PDC reactors.

Preferably, the first electrode and the second electrode are rectangular, square or “U” in shape.

According to another embodiment of the presently claimed invention, an air purification system comprises: a plurality of plasma driven catalyst (PDC) reactors in a parallel setup; wherein the PDC reactors are stacked in parallel with each other; wherein each of the PDC reactors comprises a first zigzag electrode, a second zigzag electrode, a first dielectric layer, and a first photocatalytic coating; wherein the first zigzag electrode comprises one or more first zigzag structures, and the second zigzag electrode comprises one or more second zigzag structures; wherein the first zigzag electrode and the second zigzag electrode are located in parallel with each other, and the first dielectric layer is placed between the first zigzag electrode and the second zigzag electrode and formed on the first zigzag electrode; wherein the first photocatalytic coating is coated on the second electrode or the first dielectric layer.

Preferably, the first zigzag electrode and the zigzag second electrode are rectangular, square or “U” in shape.

The system of the present invention provides the advantages with ease of fabrication and scaling-up, and improvement of air purification performance. In addition, lower voltage is needed in parallel setup to achieve the same performance as the honeycomb configuration because the plasma density and discharge area are larger than honeycomb configuration. The size of parallel setup is compact comparing to the honeycomb configuration.

### BRIEF DESCRIPTION OF THE DRAWINGS:

Embodiments of the present invention are described in more detail hereinafter with reference to the drawings, in which:

FIG. 1 is a schematic diagram showing a plasma driven catalyst disinfecting and purifying apparatus according to an embodiment of the presently claimed invention;

FIG. 2 is a schematic diagram showing a plasma reactor in a PDC apparatus with catalyst coated on electrodes according to an embodiment of the presently claimed invention;

FIG. 3 is a schematic diagram showing a plasma reactor in a PDC apparatus with catalyst located between two electrodes according to an embodiment of the presently claimed invention;

FIG. 4 is a schematic diagram showing a plasma reactor in a PDC apparatus with catalyst located at back end of the plasma reactor according to an embodiment of the presently claimed invention;

FIG. 5 is a photo showing an experimental set-up for an air pollutant removal test according to an embodiment of the presently claimed invention;

FIG. 6 is a schematic diagram showing a top view of an air purification system with a honeycomb configuration according to an embodiment of the presently claimed invention;

FIG. 7A is a schematic diagram showing a configuration of multiple PDC reactors in a parallel setup with one dielectric layer between two electrodes according to an embodiment of the presently claimed invention;

FIG. 7B is a schematic diagram showing a configuration of multiple PDC reactors in a parallel setup with two dielectric layers between two electrodes according to an embodiment of the presently claimed invention;

FIG. 8A is a schematic diagram showing a configuration of multiple PDC reactors in a parallel setup with one dielectric layer between two zigzag electrodes according to an embodiment of the presently claimed invention;

FIG. 8B is a schematic diagram showing a configuration of multiple PDC reactors in a parallel setup with two dielectric layers between two zigzag electrodes according to an embodiment of the presently claimed invention;

FIG. 9A is a plan view showing an electrode in “U” shape according to an embodiment of the presently claimed invention; and

FIG. 9B is another view showing the electrode of FIG. 9A.

### DETAILED DESCRIPTION OF PREFERRED EMBODIMENTS

In the following description, a plurality of air purification apparatuses and systems are set forth as preferred examples. It will be apparent to those skilled in the art that modifications, including additions and/or substitutions, may be made without departing from the scope and spirit of the invention. Specific details may be omitted so as not to obscure the invention; however, the disclosure is written to enable one skilled in the art to practice the teachings herein without undue experimentation.

The plasma driven catalyst disinfecting and purifying apparatus in this invention can remove air pollutants and improve indoor air quality effectively and efficiently. This apparatus comprises a pre-filter, an electric fan, and a plasma reactor with catalyst inside. The plasma reactor is based on dielectric barrier discharge (DBD) plasma, which comprises two parallel spaced electrodes, and one or two dielectric barriers. The electrode is made of electrically conductive materials which may be in form of rods, tubes, pipe, foils, films, plates, or mesh. The distance between the two electrodes ranges from a few millimeters to one hundred millimeter. The electrodes are separated by the dielectric barriers and these barriers are either attached to the electrodes or inserted between two electrodes. A high voltage alternating current (AC) from 4 kV to 30 kV with the frequency ranging from several hundred hertz (Hz) to a few hundred kilo hertz (kHz) is applied on the electrodes to generate the DBD plasma inside the reactor.

The combination of plasma and catalyst for air treatment has many advantages, such as higher energy efficiencies, low power consumption, high mineralization rates and absence of by-product formation. This plasma driven catalytic air cleaning technology enables deep purification by decomposing a whole range of toxic compositions into CO₂ and H₂O at low temperature. Changing plasma characteristics can eventually result in enhancing the production of new active species and increasing the oxidizing power of the plasma discharge. Plasma discharges also affect catalyst properties such as a change in chemical composition, enhancement in surface area, or change of catalytic structure. The catalyst in the plasma zone is activated by the plasma and the activation mechanisms include ozone, UV, local heating, changes in work function, activation of lattice oxygen, adsorption/desorption, creation of electron-hole pairs, and direct interaction of gas-phase radicals with adsorbed pollutants. Besides assisting to degrade the gas pollutants in the plasma reactor, the activated catalyst can also degrade the toxic by-products generated from the plasma. Thus, this plasma driven catalyst technology has much higher air purification efficiency and lower toxic by-products emission than using plasma only, or other air purification technologies.

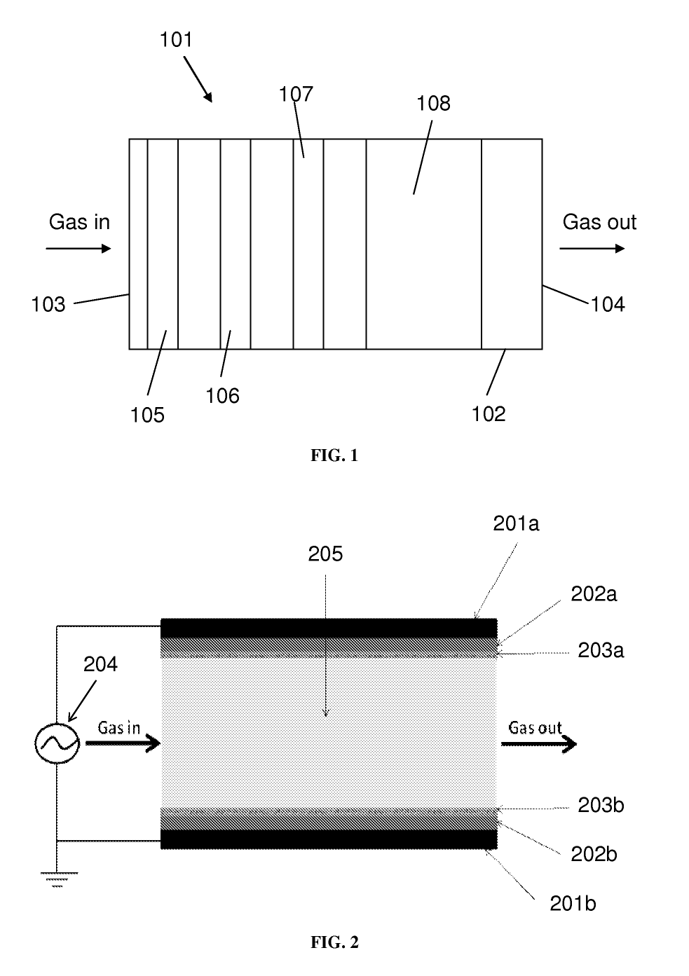

FIG. 1 is a schematic diagram showing a plasma driven catalyst disinfecting and purifying apparatus according to an embodiment of the presently claimed invention. The apparatus 101 comprises a casing 102 having an air inlet 103, and an air outlet 104, an electric fan 105, an orientation air deflector 106, a pre-filter 107, and a plasma reactor 108. The casing 102 encloses the electric fan 105, the orientation air deflector 106, the filter 107, and the plasma reactor 108. The electric fan 105 generates airflow. The orientation air deflector 106 orientates the direction of the airflow. The pre-filter 107 removes air particulates. The plasma reactor 108 generates plasma for disinfecting and purifying air.

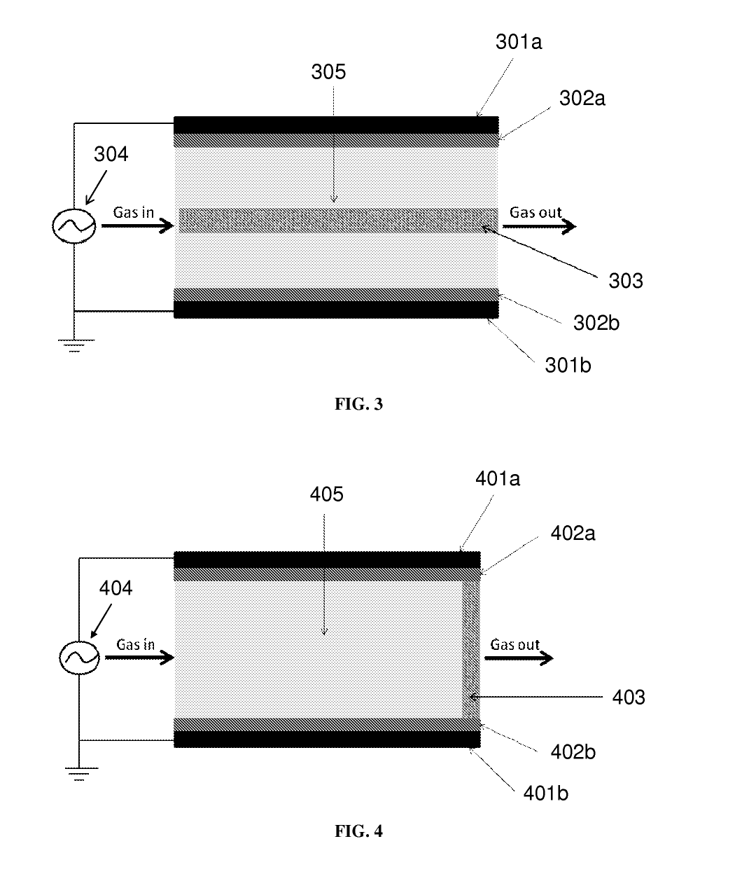

FIG. 2 is a schematic diagram showing a plasma reactor in a PDC apparatus with catalyst coated on electrodes according to an embodiment of the presently claimed invention. The plasma reactor comprises a pair of spaced plasma electrodes, 201a and 201b, two insulating dielectric layers 202a and 202b, two photocatalyst layers, 203a and 203b, an AC power supply 204, an air inlet for gas in and an air outlet for gas out. The spaced plasma electrodes 201a and 201b are positioned in parallel with each others with a distance. The insulting dielectric layer 202a is positioned on the spaced plasma electrode 201a and in face of the spaced plasma electrode 201b. Similarly, the insulting dielectric layer 202b is positioned on the spaced plasma electrode 201b and in face of the spaced plasma electrode 201a. The photocatalyst layer 203a is coated on the insulting dielectric layer 202a, and the photocatalyst layer 203b is coated on the insulting dielectric layer 202b. Such that the photocatalyst layer 203a is in face of the spaced plasma electrode 201b while the photocatalyst layer 203b is in face of the spaced plasma electrode 201a. When the AC power supply 204 provides high voltage alternating current to the spaced plasma electrodes 201a and 201b, a plasma 205 is generated within a plasma zone located between the spaced plasma electrodes 201a and 201b. Both of the photocatalyst layers 203a and 203b are in contact with the plasma 205. When polluted air from the air inlet passes through the plasma 205 in the plasma reactor, the polluted air is purified and disinfected, and the purified air is released out from the air outlet.

Since the photocatalyst layers are directly coated on the insulting dielectric layers, the photocatalyst layers can be effectively activated by the plasma in the plasma reactor without additional UV light irradiation to generate free radicals, which enable to decompose air pollutants such as VOC into non-harmful products like water and carbon dioxide, thereby further enhancing the air pollutant removal efficiency. Since the photocatalyst is in contact with the plasma, the efficiency of free radical generation is further increased under such reactive plasma environment. In addition, ozone or other harmful byproducts generated from the plasma are also eliminated by the free radicals.

As the photocatalyst layers are coated on the insulating layers, nearly no air resistance is generated from the photocatalyst layers, ultimately sustaining high airflow condition and reducing the burden of the electric fan of the air purification apparatus.

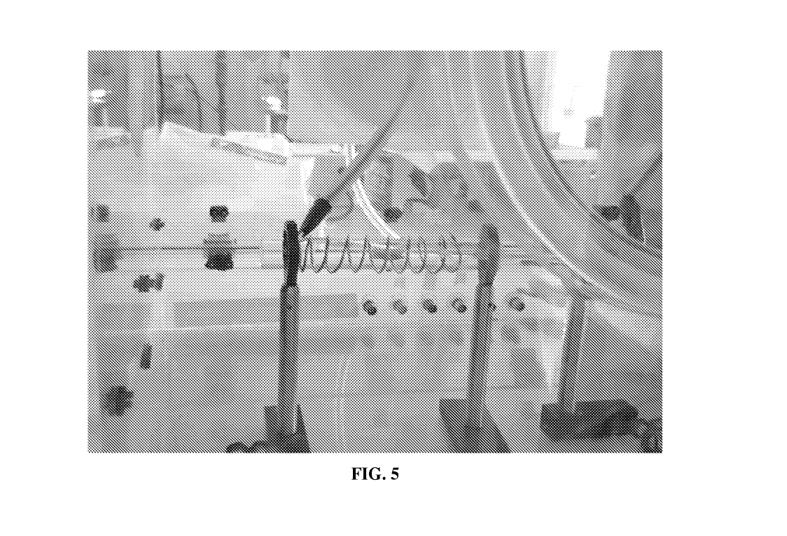

FIG. 3 is a schematic diagram showing a plasma reactor in a PDC apparatus with catalyst located between two electrodes according to an embodiment of the presently claimed invention. In this embodiment, a photocatalyst layer 303 is located in a plasma zone between a pair of plasma spaced electrodes, 301a and 301b, and placed in substantially parallel with the pair of plasma spaced electrodes, 301a and 301b. The photocatalyst layer 303 is immersed and in contact with a plasma 305 generated by the pair of the plasma spaced electrodes such that the photocatalyst layer 303 is effectively activated by the plasma 305 to generate free radicals for decomposing air pollutants and eliminating ozone and other harmful by-products released from the plasma 305 without additional UV light irradiation. In addition, as the photocatalyst layer 303 is positioned in parallel along with the airflow, nearly no air resistance is generated from the photocatalyst layer 303. Similarly, insulating dielectric layers 302a and 302b are coated on the pair of plasma spaced electrodes, 301a and 301b respectively. An AC voltage is provided to the electrodes by an AC power supply 304 connected to the electrodes.

Preferably, the photocatalyst layer has a thickness ranging of from 10 μm to 500 μm. The insulating dielectric layer has a thickness ranging of 1 mm to 5 mm.

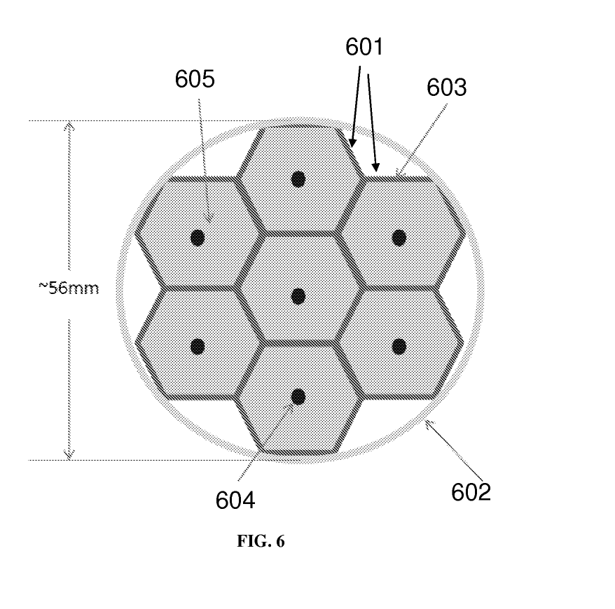

FIG. 4 is a schematic diagram showing a plasma reactor with catalyst located at back end of the plasma reactor according to an embodiment of the presently claimed invention. In this embodiment, a photocatalyst layer 403 is located at the back end of the plasma reactor, and covers the air outlet of the plasma reactor. The surface of the photocatalyst layer 403 is exposed to a plasma zone between a pair of plasma spaced electrodes, 401a and 401b, and in contact with a plasma 405 such that the photocatalyst layer 403 is effectively activated by the plasma 405 to generate free radicals for decomposing air pollutants and eliminating ozone and other harmful by-products released from the plasma 405 without additional UV light irradiation. Such configuration can provide better air pollutant removal efficiency. Similarly, insulating dielectric layers 402a and 402b are coated on the pair of plasma spaced electrodes, 401a and 401b respectively. An AC voltage is provided to the electrodes by an AC power supply 404 connected to the electrodes.

Preferably, a TiO₂-based coating is incorporated in the reactor. This catalyst has a plurality of mesoporous structures with a pore size of 2-20 nm with the increased total effective surface area. The TiO₂ catalyst may be doped with other elements, such as Ti, Zn, Cu, Mn, La, Mo, W, V, Se, Ba, Ce, Sn, Fe, Mg, Au, Pt, Co, Ni, or Pd, or its oxides to enhance its photocatalytic performance. This catalyst can be coated on the dielectric barriers or other substrates, such as air permeable substrate, metal, glass, ceramic, plastic, and fabric. The position of the catalyst can be on the surface of the electrodes, between electrodes, or at the back end or front end of the plasma reactor.

Preferably, the sol-gel method is used to coat the catalyst on the dielectric layer. The precursor of the photocatalyst with other chemicals is mixed well to form a pre-photocatalyst solution. Then the coating is formed on the dielectric layer by dip coating. After that, the coating is annealed in a furnace to form the photocatalyst layer.

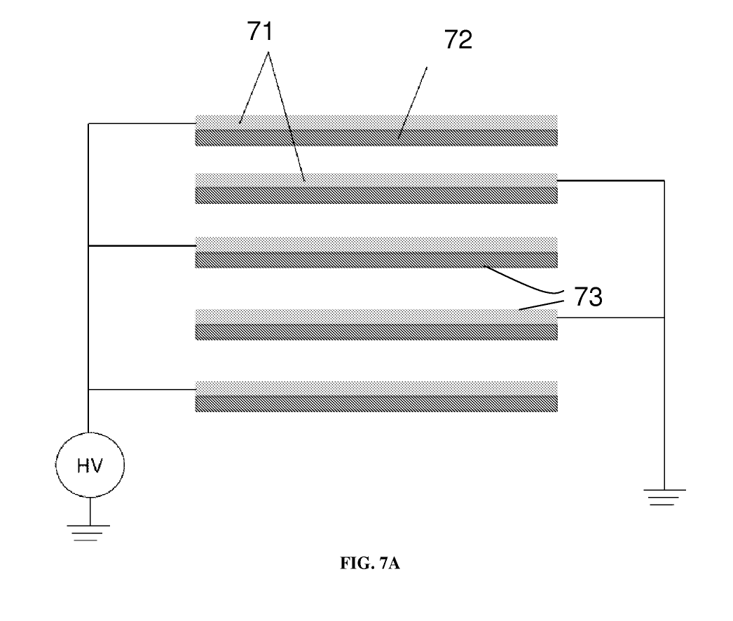

An experiment was conducted to study the air pollutant removal efficiency with a tubular plasma reactor of the present invention. As shown in FIG. 5, the plasma reactor comprised a dielectric tube with an internal diameter of 14 mm, serving as a dielectric insulating layer. TiO₂ was coated on the inner surface of the dielectric tube. A wire was coiled on the outer surface of the dielectric tube as a grounding electrode, and another wire was positioned at the center of the dielectric tube as a high voltage electrode. Both of the electrodes were connected with an AC power supply for providing alternating current towards the electrodes. The plasma reactor was placed in a closed box, in which there was an electric fan for generating airflow. VOC solution was then injected into the closed box, and the concentration of the VOC in the closed box was monitored by a VOC monitor. When an initial VOC concentration reached to a maximum value, the plasma reactor was switched on to generate plasma for purifying air in the closed box. After 30 min, a final VOC concentration was measured to calculate the air pollutant removal efficiency. The tests were repeated with three different AC voltages. A control experiment was conducted by a plasma generator without TiO₂ coating, which served as a conventional air purifier with plasma technology only.

Table 1 shows the test results conducted by the plasma reactor of the present invention.

TABLE 1

| | | | Initial | Final | |
|---|---|---|---|---|---|
| Voltage | Freq. | Time | VOC | VOC | Efficiency |
| (kV) | (kHz) | (min) | (ppm) | (ppm) | (%) |
| 18 | 6 | 30 | 28 | 15 | 46% |
| 15.2 | 6 | 30 | 27 | 19 | 30% |
| 12 | 6 | 30 | 28 | 23.5 | 16% |

Table 2 shows the test results of the control experiment.

TABLE 2

| | | | Initial | Final | |
|---|---|---|---|---|---|
| Voltage | Freq. | Time | VOC | VOC | Efficiency |
| (kV) | (kHz) | (min) | (ppm) | (ppm) | (%) |
| 18 | 6 | 30 | 27 | 18 | 33% |
| 15.2 | 6 | 30 | 27.5 | 22.5 | 18% |
| 12 | 6 | 30 | 28.5 | 26 | 9% |

As shown from the results, the plasma generator of the present invention provides much higher air pollutant removal efficiencies under the three voltages than those of the control experiment. After incorporating the photocatalyst layer on the insulating dielectric layer, the removal efficiencies are substantially increased in a range of 39% to 78%.

An ozone removal test was conducted with the above set-up. Ozone monitor was used to measure ozone concentration in the closed box. Two experiments were conducted with the plasma reactor of the present invention (with photocatalyst coating), and a plasma reactor without photocatalyst coating (control experiment) respectively. Initial ozone concentration was measured before switching on the plasma reactor, and final ozone concentration was measured after 30 min. The results are shown in Table 3.

TABLE 3

| | Voltage | Freq. | Time | Initial O₃ | Final O₃ |
|---|---|---|---|---|---|
| | (kV) | (kHz) | (min) | (ppb) | (ppb) |
| Without | 12 | 6 | 30 | 3 | 362 |
| photocatalyst | | | | | |
| coating | | | | | |
| With | 12 | 6 | 30 | 5 | 5 |
| photocatalyst | | | | | |
| coating | | | | | |

As shown from the results, when there was no photocatalyst coating, the ozone concentration substantially increased from 3 ppb to 362 ppb after 30 min. In stark contrast, the ozone concentration remained the same with 5 ppb after 30 min. The results show that the plasma reactor of the present invention is capable of avoiding the release of harmful products generated by the plasma since the photocatalyst is able to remove the harmful products by generation of free radicals.

After the PDC apparatus of the present invention is assembled, the purifier can be put at a predetermined place for disinfecting and purifying indoor air.

If a higher airflow rate is needed, multiple plasma reactors can be integrated together as a honeycomb configuration to form an air purification system. This system can provide higher air purification efficiency, larger airflow rate and longer catalyst lifetime.

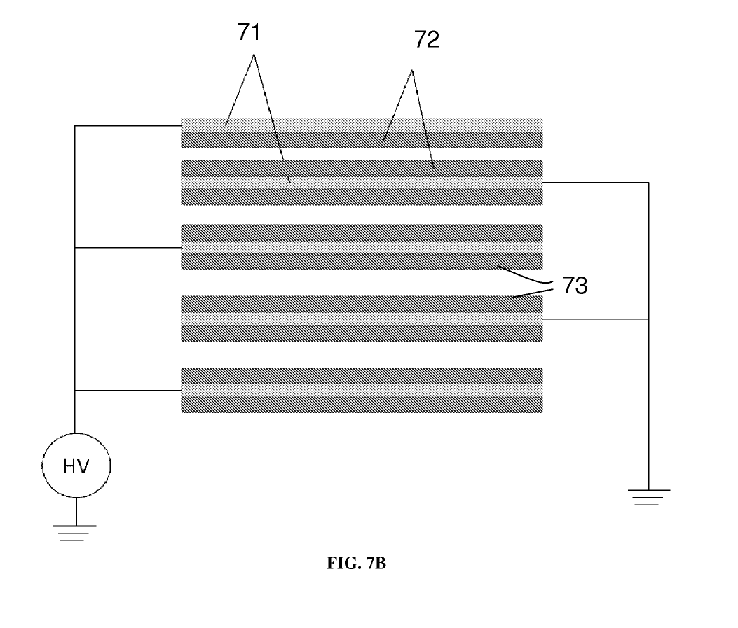

FIG. 6 is a schematic diagram showing a top view of a purification system with a honeycomb configuration according to an embodiment of the presently claimed invention. The system comprises a plurality of plasma reactors 601 of present invention, and a polyethylene terephthalate (PET) plastic shield housing 602. The PET plastic shield housing 602 is 56 mm in diameter and 10 cm in height, and the thickness of the housing 602 is 2-3 mm. Each of the plasma reactors is in a honeycomb shape so that the plasma reactors can be tightly stacked together for space saving. The plasma reactor comprises stainless steel walls 603 having a thickness of 1-2 mm serving as a grounding electrode, and a high voltage electrode 604. There are TiO₂ coating and dielectric coating formed on the inner side of the stainless steel walls 603. When an AC power is applied, a plasma 605 is generated between the grounding electrode 603 and the high voltage electrode 604. Since the total plasma zone is substantially increased under such configuration, it is more effective for the air purification system to remove air pollutants.

The present invention is applicable for indoor air quality improvement in domestic and industrial air treatment environment, such as city hall and buildings, airports and train stations, public smoking rooms, underground malls, health care centers, clean manufacturing sites, etc.

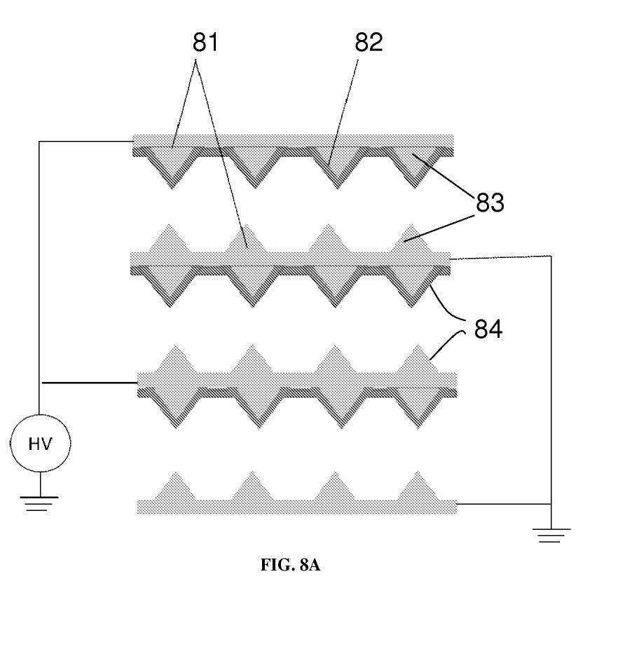

FIG. 7A is a schematic diagram showing a configuration of multiple PDC reactors in a parallel setup with one dielectric layer between two electrodes according to an embodiment of the presently claimed invention. The PDC reactor comprises two electrodes 71 and one dielectric layer 72. The electrodes 71 are placed in parallel with each other. The dielectric layer 72 is located between the two electrodes 71 and formed on one of the electrodes 71. Two photocatalytic coatings 73 are coated on both the electrode 71 and the dielectric layer 72 respectively. However, the photocatalytic coating 73 can be coated on the electrodes 71 or the dielectric layers 72 only.

Preferably, a gap between the first dielectric layer and the second electrodes is 1-10 mm. An alternating current power supply connecting to the first and the second electrodes provides a voltage of 5-20 kV at 5-25 kHz.

A cigarette smoke removal test was conducted with the configuration of FIG. 7A. The configuration comprises five PDC reactors. The applied AC voltage was 10 kV at 6 kHz. The gap between each dielectric layer and each electrode is 2 mm and the electrode area is 2.5 cm². The configuration removed 18 ppm VOCs from the cigarette smoke in one passage way with the gas flow rate 1 m/s.

FIG. 7B is a schematic diagram showing a configuration of multiple PDC reactors in a parallel setup with two dielectric layers between two electrodes according to an embodiment of the presently claimed invention. The configuration of FIG. 7B is similar to that of FIG. 7A, but there are two dielectric layers 72 located between two electrodes 71. The photocatalytic coatings 73 are coated on the dielectric layers 72.

Comparing with a honeycomb PDC reactor, the configuration of multiple PDC parallel reactors of the present invention provides several advantages as below:

1. The parallel setup is easier to be fabricated and scaled up than the honeycomb configuration.

2. Lower voltage is needed in parallel setup to achieve the same performance as the honeycomb configuration because the plasma density and discharge area are larger than honeycomb configuration.

3. The size of parallel setup is compact comparing with the honeycomb configuration.

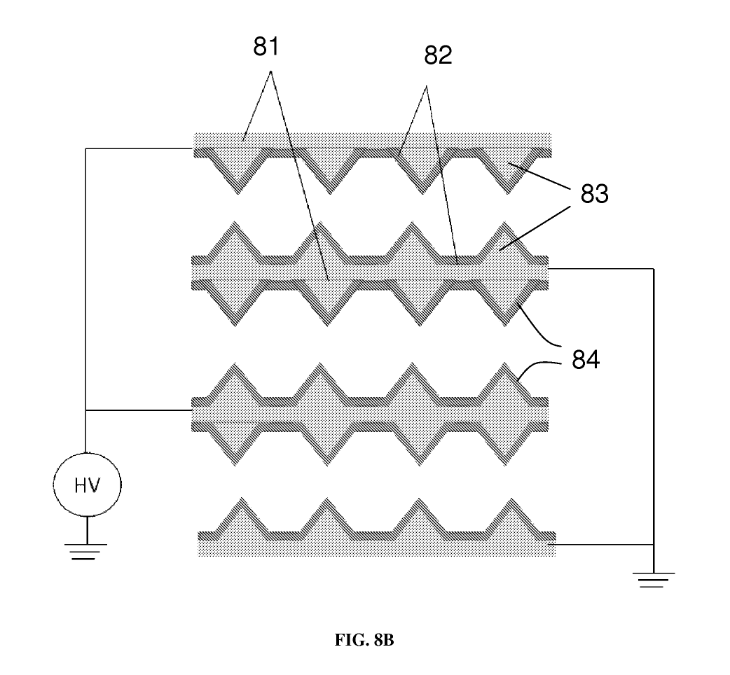

FIG. 8A is a schematic diagram showing a configuration of multiple PDC reactors in a parallel setup with one dielectric layer between two zigzag electrodes according to an embodiment of the presently claimed invention. The PDC reactor comprises two zigzag electrodes 81 and one dielectric layer 82. Each of the zigzag electrodes 81 comprises a plurality of zigzag structures 83, which are capable of facilitating the plasma generation and enhancing plasma density. The zigzag electrodes 81 are place in parallel with each other. The dielectric layer 82 is located between the two zigzag electrodes 81, and formed on one of the zigzag electrodes 81. The photocatalytic coatings 84 are coated on both of the zigzag electrode 81 and the dielectric layer 82. However, the photocatalytic coatings 84 can be coated on the zigzag electrode 81 or the dielectric layers 82 only. Apart from the zigzag electrodes 81, wavy electrodes can be used, which comprise a plurality of wavy structures.

Comparing with the configuration of FIG. 7A, the configuration of FIG. 8A can provide the advantage of smaller air resistance since the plasma generated is concentrated nearby the region of the zigzag structures.

FIG. 8B is a schematic diagram showing a configuration of multiple PDC reactors in a parallel setup with two dielectric layers between two zigzag electrodes according to an embodiment of the presently claimed invention. The configuration of FIG. 8B is similar to that of FIG. 8A, but there are two dielectric layers 82 located between two electrodes 81. The photocatalytic coatings 83 are coated on the dielectric layers 82.

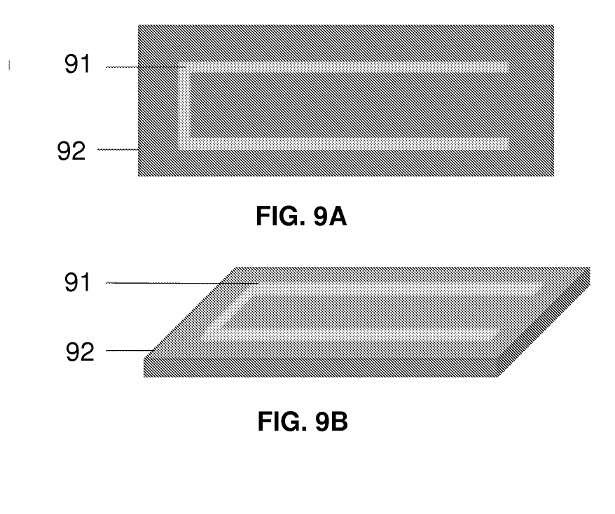

The electrodes of the PDC reactor can be rectangular or square in shape but the “U” shape can be implemented as well. As shown in FIGS. 9A and 9B, an “U” shaped electrode 91 is formed on a rectangular dielectric layer 92. The advantage provided by the “U” shaped electrode is that since the plasma generated is concentrated nearby the areas of the electrode, this configuration provides more spaces without plasma, thereby reducing the air resistance come from the plasma and enhancing the airflow rate. Ultimately, the air purification efficiency is increased.

The foregoing description of the present invention has been provided for the purposes of illustration and description. It is not intended to be exhaustive or to limit the invention to the precise forms disclosed. Many modifications and variations will be apparent to the practitioner skilled in the art.

The embodiments were chosen and described in order to best explain the principles of the invention and its practical application, thereby enabling others skilled in the art to understand the invention for various embodiments and with various modifications that are suited to the particular use contemplated. It is intended that the scope of the invention be defined by the following claims and their equivalence.

What is claimed is:

1. An air purification system, comprising:
a plurality of plasma driven catalyst (PDC) reactors in a parallel setup;
wherein the PDC reactors are stacked in parallel with each other;
wherein each of the PDC reactors comprises a first electrode, a second electrode, a first dielectric layer, and a first photocatalytic layer;
wherein the first electrode and the second electrode are located in parallel with each other, and the first dielectric layer is placed between the first electrode and the second electrode and formed on the first electrode;
wherein the first photocatalytic coating is coated on the second electrode or the first dielectric layer.

2. The air purification system of claim 1, wherein the PDC reactors are rectangular or square in shape for facilitating stacking among the PDC reactors.

3. The air purification system of claim 1, wherein the first electrode and the second electrode are rectangular, square or “U” in shape.

4. The air purification system of claim 1, wherein one of the first electrode and the second electrode comprises one or more zigzag structures or one or more wavy structures.

5. The air purification system of claim 1, wherein the each of the PDC reactors further comprises a second photocatalytic coating, and the first photocatalytic coating is coated on the first dielectric layer, and the second photocatalytic coating is coated on the second electrode.

6. The air purification system of claim 1, wherein the each of the PDC reactors further comprises a second dielectric layer placed between the first electrode and the second electrode and formed on the second electrode.

7. The air purification system of claim 6, wherein the each of the PDC reactors further comprises a second photocatalytic coating, and the first photocatalytic coating is coated on the first dielectric layer, and the second photocatalytic coating is coated on the second dielectric layer.

8. The air purification system of claim 1, wherein a gap between the first dielectric layer and the second electrodes is in a range of 1 to 10 mm.

9. The air purification system of claim 1, further comprising:
an alternating current power supply connecting to the first and the second electrodes of the each of the PDC reactors.

10. The air purification system of claim 1, wherein the alternating current power supply is used for providing a voltage of 5-20 kV at 5-25 kHz.

11. An air purification system, comprising:
a plurality of plasma driven catalyst (PDC) reactors in a parallel setup;
wherein the PDC reactors are stacked in parallel with each other;
wherein each of the PDC reactors comprises a first zigzag electrode, a second zigzag electrode, a first dielectric layer, and a first photocatalytic coating;
wherein the first zigzag electrode comprises one or more first zigzag structures, and the second zigzag electrode comprises one or more second zigzag structures;
wherein the first zigzag electrode and the second zigzag electrode are located in parallel with each other, and the first dielectric layer is placed between the first zigzag electrode and the second zigzag electrode and formed on the first zigzag electrode;
wherein the first photocatalytic coating is coated on the second electrode or the first dielectric layer.

12. The air purification system of claim 11, wherein the PDC reactors are rectangular or square in shape for facilitating stacking among the PDC reactors.

13. The air purification system of claim 11, wherein the first zigzag electrode and the zigzag second electrode are rectangular, square or “U” in shape.

14. The air purification system of claim 11, wherein the each of the PDC reactors further comprises a second photocatalytic coating, and the first photocatalytic coating is coated on the first dielectric layer, and the second photocatalytic coating is coated on the second electrode.

15. The air purification system of claim 11, wherein the each of the PDC reactors further comprises a second dielectric layer placed between the first zigzag electrode and the second zigzag electrode and formed on the second zigzag electrode.

16. The air purification system of claim 15, wherein the each of the PDC reactors further comprises a second photocatalytic coating, and the first photocatalytic coating is coated on the first dielectric layer, and the second photocatalytic coating is coated on the second dielectric layer.

17. The air purification system of claim 11, further comprising:
an alternating current power supply connecting to the first and the second electrodes of each of the PDC reactors.

18. A plasma driven catalyst reactor, comprising:
at least one zigzag electrode comprising one or more zigzag structures or one or more wavy structures;
a dielectric layer formed on the zigzag electrode; and
a photocatalytic coating coated on the dielectric layer.
